(19) United States
(12) Patent Application Publication
(10) Pub. No.: US 2003/0165433 A1
(43) Pub. Date: Sep. 4, 2003

(54) Throat spray for stopping hiccups

(76) Inventor: Hussein Amr, Northville, MI (US)

Correspondence Address: John Chupa & Associates P C, 28535 Orchard Lake Road, Suite 50, Farmington HIlls, MI, 48334, US

(21) Appl. No.: 10/095,806

(22) Filed: Mar. 12, 2002

Related U.S. Application Data
(63) Continuation-in-part of application No. 09/575,482, filed on May 19, 2000, now abandoned.

(51) Int. Cl. A61L 9/04; A61K 35/78

(52) U.S. Cl. 424/45; 424/736; 222/402.1

(57) ABSTRACT

A throat spray for stopping hiccups. The throat spray includes a liquid compound 40 which is operatively contained within a spray bottle 20. The spray bottle 20 selectively discharges the compound 40 into a user's throat in a finely atomized spray, effective to stop hiccups.

This is a continuation-in-part of pending U.S. patent application Ser. No. 09/575,482, filed May 19, 2000.

### FIELD OF INVENTION

The present invention generally relates to a throat spray, and more specifically to a throat spray for use in stopping hiccups.

### BACKGROUND OF THE INVENTION

Hiccups are a common human physical condition occurring with varying frequency and severity between individuals. The condition is reactive in nature and is a function of repeated involuntary spasmodic contractions of the diaphragm. The diaphragm contraction is followed by a sudden closure of the glottis, which abruptly cuts the incoming airflow resulting in a characteristically familiar sound. While it is understood how various nerve stimulations affect these spasmodic episodes of the diaphragm, the actual mechanism of causation is generally unknown. It is, however, common for certain gastric upsets to bring on the condition, such as, moderate to excessive alcohol consumption or overeating.

Typically, an occurrence of hiccups is temporary in nature. Yet, even if the event is not severe and short-lived, hiccups are often disrupting and uncomfortable to the individual. A prolific number of “home remedies” have been offered to hiccups sufferers over the years. Most people are somewhat familiar with the home remedy approach in attempted hiccup cessation. These home remedies vary from the simplistic to the ridiculous. All of which generally fail in their design purpose or are exceedingly slow to act.

Of concern, continued or severe hiccups can escalate to a harmful level. There are doctor prescribed medications available that act on the nerves influencing the diaphragm to stop the spasms. In the most severe cases, surgery has been performed to sever the phrenic nerve connection with the diaphragm. The drawbacks to these professional medical remedies are obvious. Doctor visits and prescription medicines are expensive, and surgery carries its own risks, is highly invasive, and costly.

It is therefore desirable to offer an effective, low cost, and fast acting remedy to stop hiccups.

### SUMMARY OF THE INVENTION

It is a first object of the present invention to provide an effective, low cost throat spray to stop hiccups.

According to a first aspect of the present invention, an apparatus is provided. The apparatus includes a spray bottle and a liquid compound. The liquid compound is selectively discharged and atomized by the spray bottle. The compound is of natural ingredients, non-toxic and safe to consume. In one non-limiting embodiment, the compound is comprised of lemon extract and water, and has been effective in test groups for providing relief from hiccups. The compound can be made readily available to the consumer over-the-counter, without the need for FDA approval or prescription. The compound can be configured to be offered at a relative cost comparable with other types of over-the-counter products, such as cold and flu preparations. The liquid compound provided is provided in a hand held atomizing spray bottle which may include a pre-pressurized gas. The compound acts in a most expedient manner, as demonstrated in test groups when the compound is delivered as an atomized spray mist directly to the back of the throat.

These and other features, aspects, and advantages of the invention will become apparent by reading the following specification and by reference to the following drawings.

### BRIEF DESCRIPTION OF THE DRAWINGS

FIG. 1 is a perspective view of the invention used in topical delivery of the provided liquid to the throat of the user, and;

FIG. 2 is a perspective view of the invention being used.

### DETAILED DESCRIPTION OF THE PREFERRED EMBODIMENT OF THE INVENTION

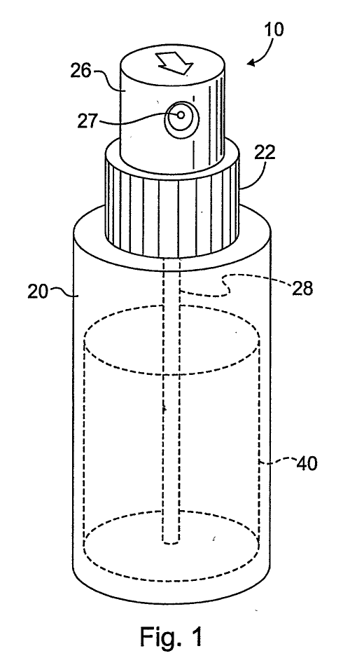

Referring now to FIG. 1, there is an apparatus 10 for stopping hiccups. Apparatus 10 comprises a spray bottle 20 and a liquid compound 40, which is selectively discharged and atomized by the spray bottle 20.

Liquid compound 40 is a non-toxic, natural mixture comprising lemon juice, concentrate, and/or extract. In the preferred embodiment, liquid compound 40 is a mixture of a commercially available lemon extract and purified water. The lemon extract used in the preferred embodiment is produced and available through Northville Laboratories of Northville, Mich., and is sold under the trade name of “F1505 lemon extract”.

The liquid compound 40 of “F1505 lemon extract” mixed with purified water is effective in varying concentrations. For example and without limitation, in one non-limiting embodiment, the compound may vary between 6:1 parts extract to water to 2:1 parts extract to water. It should be understood that the described embodiment is non-limiting, and that variations in the supplier and the exact formulation of the extract and the mixed compound may be utilized in alternate embodiments of the invention.

In one non-limiting embodiment, the spray bottle 20 is a manual pump action bottle with a screw type top 22 containing a conventional internal pump assembly (not shown), and a spray nozzle tip 26. In the preferred embodiment, spray bottle 20 is a small hand-held bottle that can be easily inserted into and/or stored within a user's purse or pocket. The internal pump assembly is fed by a pickup tube 28, which is attached to the pump assembly and is suspended into the open cavity of the supply bottle 20 and thus into the liquid compound 40.

The spray nozzle tip 26 is operatively disposed on top of the internal pump assembly, such that, when the spray nozzle tip 26 is depressed by the finger of a user, the internal pump assembly is depressed, which in turn collapses an internal cavity within the pump assembly.

When the spray nozzle tip 26 is depressed by the finger of a user for the very first time no liquid compound is discharged. However, when the spray nozzle tip 26 is released, the internal pump assembly returns to its undepressed position by an internal spring action. As the internal pump assembly moves upwards, the expansion in its internal cavity creates a suction which draws a portion of the liquid compound 40 contained in the supply bottle 20 up through the pick up tube 28. The drawn liquid compound 40 remains within the expanded internal cavity of the pump assembly until the spray nozzle tip 26 is again depressed.

Then, when the spray nozzle tip 26 and internal pump assembly are depressed by the finger of the user the second time and any time thereafter, an internal cavity within the pump assembly collapses. Since the cavity of the internal pump assembly has been filled with the liquid compound 40 from the spray bottle 20 by the previous depression of the spray tip 26, this stored portion of the liquid compound 40 is forced out through the atomizing opening 27 of the nozzle tip 26. This creates a propelled spray or mist of the liquid compound 40.

Then as the spray nozzle tip 26 is released, the internal cavity of the pump assembly is refilled with the drawn liquid compound 40 awaiting the next pump action as described above.

It should be understood that the described embodiment is non-limiting and variations in the delivery method of a spray bottle do not depart from the spirit or scope of the invention. For example and without limitation, other embodiments may include the liquid compound 40 contained within a pressurized spray container, and more particularly a sealed aerosol type can. In this embodiment, the internal cavity of the bottle would contain the liquid compound 40 and a pre-pressurized, non-toxic, propellant type gas (e.g. nitrous oxide, carbon dioxide) which selectively forces the compound 40 in an atomized and/or vaporized form through a spray nozzle when activated.

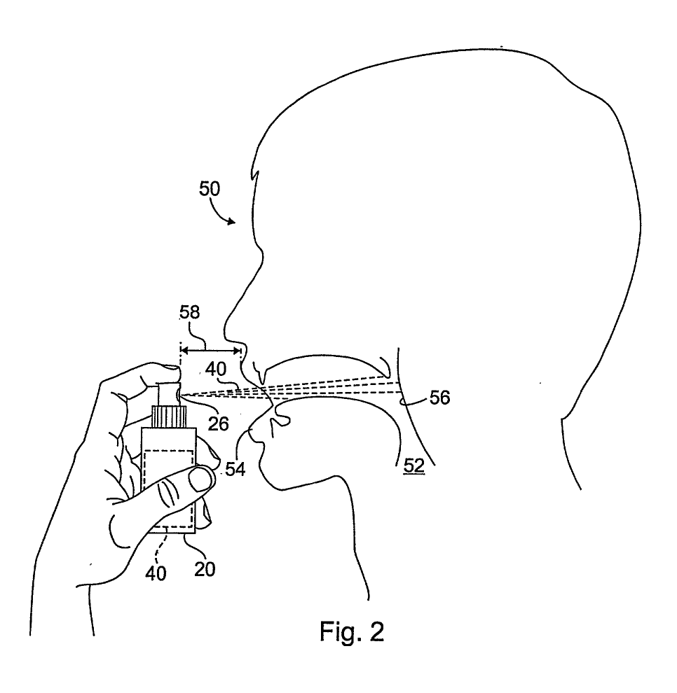

Referring now to FIG. 2, in operation, a user 50 selectively depresses or activates the spray nozzle tip 26 of the spray bottle 20 in order to apply the liquid compound 40 to the back of the user's throat 56 in a finely atomized spray. Specifically, a user so directs, or aims the spray nozzle tip opening 27 of the spray bottle 20 into his or her open mouth 54, and more particularly, at the back of the user's throat 56. The user then selectively depresses the spray nozzle tip 26, thereby delivering the compound 40 into the user's throat 56 (e.g., to the back of the user's throat).

The optimum distance 58 between the spray nozzle tip 26 of the spray bottle 20 and the mouth 54 of the user 50 is approximately two inches or less. The user may vary this distance but effectiveness of the delivery of the liquid compound to the back of the user's throat 52 diminishes as the distance increases. It has been found that the fine atomized spray that is provided by the preferred embodiment of the present invention and which directly delivers the said compound to the back of the user's throat, provides substantially improved results over other methods of delivery (e.g., by drinking the mixture). In yet another alternate embodiment of the invention, The principles of the present invention may be applied with particular advantage to obtain a spraying device containing a mixture of limejuice from Concentrate (9.0% by volume), Terpenless oil (1.0% by volume), and Lemon Juice from Concentrate (90.0% by volume), for use in stopping hiccups. Particularly, this mixture or composition provided unexpected “hiccup stopping” or alleviation results.

A microbiological determination was made on two separate samples of the aforementioned throat spray which were two years old and the determination was made that after forty-eight hours of incubation, no bacteria colonies were observed and that the pH of the throat spray has an extremely low level of 2.5 (i.e., the solution is naturally acidic) and, because the pH of this solution is extremely low, a food preservative such as Sodium Benzoate or Sodium Bisulfate or a mixture of both may not be necessary to include within this solution, however, in one non-limiting embodiment of the present invention, a food grade preservative such as Sodium Benzoate or Sodium Bisulfate or a mixture of both may be utilized. Hence, it should be understood that nothing in this description is meant to limit this solution to include a food preservative. It should also be understood that the Sodium Benzoate which may be found in this solution is classified as a food product and, therefore cannot be considered as toxic or hazardous to human health.

The throat spray comprising the above mentioned solution was utilized in a field trial which continued for over one year. Over one hundred participants were chosen and the data gathered is displayed below in Table 1 and at least five separate participants, in each age group, were tested with the product.

TABLE 1

| | | Occurrence of | Duration of | | Adverse |
|---|---|---|---|---|---|
| Gender | Age group | Hiccups | Product Usage | Result | Effects |
| Male | Toddler to | Light to | Over 1 yr. | Successful control | None |
| | Young Adult | Moderate | | and/or elimination | |
| | (2-25 Yr.) | | | of Hiccups | |
| Male | Toddler to | Moderate to | Over 1 yr. | Successful control | None |
| | Young Adult | Heavy | | and/or elimination | |
| | (2-25 Yr.) | | | of Hiccups | |
| Female | Toddler to | Light to | Over 1 yr. | Successful control | None |
| | Young Adult | Moderate | | and/or elimination | |
| | (2-25 Yr.) | | | of Hiccups | |
| Female | Toddler to | Moderate to | Over 1 yr. | Successful control | None |
| | Young Adult | Heavy | | and/or elimination | |
| | (2-25 Yr.) | | | of Hiccups | |
| Male | Adult | Light to | Over 1 yr. | Successful control | None |
| | (26-50) | Moderate | | and/or elimination | |
| | | | | of Hiccups | |
| Male | Adult | Moderate to | Over 1 yr. | Successful control | None |
| | (26-50) | Heavy | | and/or elimination | |
| | | | | of Hiccups | |
| Female | Adult | Light to | Over 1 yr. | Successful control | None |
| | (26-50) | Moderate | | and/or elimination | |
| | | | | of Hiccups | |
| Female | Adult | Moderate to | Over 1 yr. | Successful control | None |
| | (26-50) | Heavy | | and/or elimination | |
| | | | | of Hiccups | |
| Male | Middle Aged | Light to | Over 1 yr. | Successful control | None |
| | to Senior | Moderate | | and/or elimination | |
| | Citizen | | | of Hiccups | |
| | (51 yr. +) | | | | |
| Male | Middle Aged | Moderate to | Over 1 yr. | Successful control | None |
| | to Senior | Heavy | | and/or elimination | |
| | Citizen | | | of Hiccups | |
| | (51 yr. +) | | | | |
| Female | Middle Aged | Light to | Over 1 yr. | Successful control | None |
| | to Senior | Moderate | | and/or elimination | |
| | Citizen | | | of Hiccups | |
| | (51 yr. +) | | | | |
| Female | Middle Aged | Moderate to | Over 1 yr. | Successful control | None |
| | to Senior | Heavy | | and/or elimination | |
| | Citizen | | | of Hiccups | |
| | (51 yr. +) | | | | |

### The Field Trial

Over one hundred men, women, and children of diverse ages participated in an independently conducted Field Trial to test the effectiveness of the throat spray. The Field Trial participants were first given a brief questionnaire which was designed to inform the laboratory of each of the individual's characteristics (i.e., the characteristics of gender, age, occurrence of hiccups, and all allergies). Trial participants were then informed about the product (i.e., throat spray) which they were to be testing, provided with instructions for correct product usage, and given instructions to document every usage of the product as well as the results from each usage.

Over one year later, the Field Trial participants returned their data which they had collected. The results were then examined by the laboratory and categorized into specific groups to be presented to the applicants.

Referring now to Table 1, there is shown a compilation of the data collected from the above-mentioned Field Trial. As shown, the categories of data include Gender, Age group, Occurrence of Hiccups, Duration of Product Usage, Result, and Adverse Effects. The grouping of data is further separated into three distinct groups which are ages two to twenty-five, ages twenty-six to fifty, and ages fifty-one and over. Each of these three distinct groups are further broken down into two sub-groups which are light to moderate occurrence of hiccups and moderate to heavy occurrence of hiccups. Moreover, each of the three age groups is further separated into gender specification (i.e., each of the three age group categories have data separated into two groups which are male and female).

The Field Trial results, as should be obvious to one who is skilled in the related art, where unanimously positive among all Field Trial participants (i.e., every man, woman, and child in each age group which was tested and every hiccup occurrence level tested for all age groups resulted in the same positive opinion of the throat spray's effectiveness). Every participant of the Field Trial concurred that the throat spray was successful in controlling and/or eliminating hiccups. Furthermore, every participant of the Field Trial reported that they did not experience any adverse effects from using the throat spray when the throat spray was used as directed.

It should be understood that each of the ingredients of the throat spray are classified as food based products and the final solution of the throat spray, which is described above, cannot be considered as toxic or hazardous to human health unless a particular individual exhibits an allergenic response to citrus fruits. It should also be understood that the embodiment of the invention which has been described is merely illustrative of one application of the principles of the invention. It should be further understood and appreciated that the described embodiment is non-limiting and variations in the supplier and the exact formulation of the extract and the mixed compound utilized do not depart from the spirit or scope of the invention.

What claimed is:

1. An apparatus for stopping hiccups comprising;
a liquid compound including lemon extract, and;
a spray bottle that selectively and operatively discharges said liquid compound into the throat of a user, thereby stopping said hiccups.

2. The apparatus as in claim 1 wherein said liquid compound further comprises purified water.

3. The apparatus as in claim 2 wherein said lemon extract comprises “F1505 lemon extract”.

4. The apparatus as in claim 1 wherein said spray bottle comprises a hand held spray bottle.

5. The apparatus as in claim 1 wherein said spray bottle comprises an aerosol spray bottle containing a pre-pressurized gas.

6. A method of stopping hiccups, comprising the steps of:
providing a liquid compound comprising lemon extract;
providing a spray bottle;
disposing said liquid compound within said spray bottle;
directing said spray bottle containing said compound inside the mouth of a user, and;
selectively activating said spray bottle effective to transfer said compound in a finely atomized spray from said bottle into the throat of a user, thereby stopping said hiccups.

7. The method of claim 6 wherein said spray bottle is held approximately two inches from the user's mouth.

8. The method of claim 6 wherein said spray bottle comprises an aerosol spray bottle.

9. The method of claim 6 wherein said spray bottle comprises a hand-held spray bottle.

10. The method of claim 6 wherein said liquid compound further comprises purified water.

11. An apparatus for stopping hiccups comprising:
a liquid solution including a certain amount of lemon extract, lemon juice from concentrate, lime juice from concentrate, and a mixture of Sodium Bisulfate and Sodium Benzoate; and
a spray bottle which selectively and operatively discharges said liquid solution into the throat of a user, thereby stopping said hiccups.

12. The apparatus as in claim 11 wherein said certain amount of lemon extract is ninety-nine one-hundredths of one percent by volume of said liquid solution and wherein said lemon extract comprises Terpenless oil.

13. The apparatus as in claim 11 wherein said certain amount of lemon juice from concentrate is ninety percent by volume of said liquid solution.

14. The apparatus as in claim 11 wherein said certain amount of limejuice from concentrate is nine percent by volume of said liquid solution.

15. The apparatus as in claim 11 wherein said mixture of Sodium Bisulfate and Sodium Benzoate is one one-hundredths of one percent by volume of said liquid solution.

16. The apparatus as in claim 11 wherein said liquid solution further comprises purified water.
